(19) United States
(12) Patent Application Publication
(10) Pub. No.: US 2006/0281174 A1
(43) Pub. Date: Dec. 14, 2006

(54) Methods for generating insulin-producing cells

(76) Inventors: Gang Xu, Belle Mead, NJ (US); John J. O'Neil JR., Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/076,548

(22) Filed: Mar. 9, 2005

Related U.S. Application Data
(60) Provisional application No. 60/551,648, filed on Mar. 9, 2004.

(51) Int. Cl. C12N 5/08; C12N 5/06

(52) U.S. Cl. 435/325; 435/366

(57) ABSTRACT

This invention relates to methods for generating insulin-producing cells. In particular, the present invention relates to methods for generating insulin-producing cells or precursors thereof in vitro or in vivo by employing mesenchymal stem cells and pancreatic cells. Insulin-producing cells or precursors thereof created in accordance with the present methods, as well as the use of these cells in the treatment of diabetes, are also provided.

### CROSS REFERENCE TO RELATED APPLICATION

This application claims priority from U.S. Provisional Application No. 60/551,648, filed on Mar. 9, 2004.

### FIELD OF THE INVENTION

This invention relates generally to methods for generating insulin-producing cells. In particular, the present invention relates to methods for generating insulin-producing cells or precursors thereof, by employing mesenchymal stem cells and pancreatic cells. The present invention also relates to insulin-producing cells or precursors thereof created in accordance with the present methods and the use of these cells in the treatment of diabetes.

### BACKGROUND

Presently there are approximately 17 million diabetic patients in the United States alone. Most cases of diabetes fall into two clinical types: type 1 (insulin-dependent diabetes mellitus or “IDDM”) and type 2 (non-insulin dependent diabetes mellitus or “NIDDM”). Approximately 10 percent of the diabetic patients are type 1 diabetics, with the remaining being type 2 diabetics.

IDDM is characterized by a partial or complete inability to produce insulin, usually due to destruction of the insulin-producing cells of the pancreatic islets of Langerhans. Absent regular insulin injections, patients with type 1 diabetes can experience a wide range of debilitating symptoms, which may progress to coma and ultimately death. Additionally, a fraction of type 2 NIDDM diabetics are insulin dependent and require insulin injections to improve their insulin resistance. Thus, both type 1 and insulin-dependent type 2 diabetics can benefit from improvements in insulin administration.

Transplantation offers an alternative method to treat diabetes. The donor tissue may be an entire pancreas, harvested from a donor, or, alternatively, isolated pancreatic islets of Langerhans. However, a major problem with transplantation has been the shortage of donor tissue. There exists, therefore, an unmet need to develop a source of insulin-producing cells displaying characteristics of a pancreatic beta cell to treat diabetes.

During embryogenesis, the pancreas is thought to develop as a result of interaction of the endoderm with the endothelium of the aorta, which may be the source of essential pancreas-inductive signals. It appears that the mesoderm may interact with the endoderm through signals or signaling molecules that induce expression of markers characteristic of the pancreatic lineage. The signals appear to direct cells in the endoderm that would otherwise develop into more rostral organs to form ectopic insulin-positive islet-like clusters.

The proliferative capacity of fully differentiated cells such as, for example beta cells is limited, and current theory suggests that the proliferative capacity of these cells decline as the cell differentiates from it's precursor phenotype into it's mature beta-cell phenotype. Large numbers of insulin-producing cells or beta cells may be produced if isolated pancreatic beta cells could be stimulated to de-differentiate, and thus regain their proliferative capacity, thus allowing for their significant expansion in vitro. Manipulation of standard culture conditions, such as, for example, oxygen and carbon dioxide concentrations, concentrations of nutrients, cell density, temperature, pH, mechanical stress and culture substrates, may promote the dedifferentiation of pancreatic cells to less differentiated cells and allow for the significant proliferation of the resulting dedifferentiated cells. It is also believed that following extensive proliferation and upon return to standard culture conditions, the undifferentiated and expanded population of cells will undergo differentiation in the presence of differentiation-inducing stimuli into insulin-producing cells, or precursors thereof.

In work leading to the present invention, the present inventors found that mesenchymal stem cells are able to promote the growth and differentiation of undifferentiated cells of pancreatic origin. The present inventors have also found that a co-culture of mesenchymal stem cells and undifferentiated pancreatic cells under appropriate culture conditions, or alternatively, co-existence of these cells within proximity in vivo, leads to the generation of insulin-producing cells or at least precursors of insulin-producing cells. Additionally, it is believed that mesenchymal stem cells, when co-cultured with cells of pancreatic origin or present within proximity of pancreatic cells in vivo, may themselves differentiate into insulin-producing cells, or precursors of insulin-producing cells.

The present invention provides methods whereby mesenchymal stem cells are employed to promote the growth and differentiation of pancreatic cells, particularly, undifferentiated cells of pancreatic origin. Prior to the present invention, there has been no evidence showing that pancreatic cells can be expanded significantly in vitro, prior to the induction of differentiation of the cells into insulin-producing cells, or precursors of insulin-producing cells. In addition, prior to the present invention, there has been no recognition that mesenchymal stem cells can promote the growth and proliferation of undifferentiated cells of pancreatic origin and further differentiation of these cells into insulin-producing cells, or precursors thereof.

### SUMMARY

The present invention generally relates to methods for generating insulin-producing cells. It has been surprisingly found by the present inventors that mesenchymal stem cells can promote the growth and differentiation of pancreatic cells, particularly, undifferentiated cells of pancreatic origin. It has also been surprisingly found by the present inventors that a co-culture of mesenchymal stem cells and pancreatic cells, particularly, undifferentiated pancreatic cells, or co-existence of these cells within proximity in vivo, can lead to the generation of glucose responsive insulin-producing cells or at least precursors of insulin-producing cells.

Accordingly, in one embodiment, the present invention provides a method of promoting the growth of pancreatic cells, particularly, undifferentiated pancreatic cells, by culturing the pancreatic cells in the presence of mesenchymal stem cells. Alternatively, a population of pancreatic cells containing undifferentiated cells may be cultured in the presence of mesenchyrnal stem cells.

In one embodiment, the present invention provides a method of promoting the growth and proliferation of undifferentiated pancreatic cells by culturing the undifferentiated pancreatic cells, or a population of pancreatic cells containing undifferentiated cells, in vitro in the presence of mesenchymal stem cells.

Another embodiment of the present invention provides a method of promoting the survival of pancreatic cells, which may be either differentiated or undifferentiated cells or a mixture thereof, by culturing the pancreatic cells in the presence of mesenchymal stem cells.

In another embodiment, the present invention provides a method of promoting the generation and/or proliferation of precursors of insulin-producing cells by obtaining undifferentiated pancreatic cells or a population of pancreatic cells containing undifferentiated cells, and culturing such cells or cell population in the presence of mesenchymal stem cells.

In still another embodiment, the present invention provides a method of generating insulin-producing cells by obtaining undifferentiated pancreatic cells, or a population of pancreatic cells containing undifferentiated cells, culturing such cells in the presence of mesenchymal stem cells to generate and/or expand precursors of insulin-producing cells, and further developing the precursors into mature insulin-producing cells.

In a further embodiment, the present invention provides a method of promoting the generation of insulin-producing cells in a mammal by administering mesenchymal stem cells to the mammal.

The insulin-producing cells as well as the precursors thereof that are generated in accordance with the present methods form another embodiment of the present invention.

In another embodiment, the present invention further provides a method of treating a diabetic subject by administering insulin-producing cells or precursors thereof produced in accordance with the methods of the present invention.

In yet another embodiment, the present invention further provides a method of treating a subject predisposed to develop diabetes, by administering insulin-producing cells, or precursors thereof produced in accordance with the methods of the present invention.

In still another embodiment, the present invention provides a method of treating a diabetic subject by administering mesenchymal stem cells to the subject, thereby promoting the generation and/or proliferation of insulin-producing cells in the subject.

In yet a further aspect of the present invention, insulin-producing cells are generated in a subject that is predisposed to develop diabetes by the administration of mesenchymal stem cells.

In a further aspect, the present invention provides a method of promoting the generation of insulin-producing cells in a mammal by administering mesenchymal stem cells to the mammal.

### DETAILED DESCRIPTION OF THE INVENTION

The pancreatic cells and mesenchymal stem cells employed in the methods of the present invention may be of any mammalian origin. The term “mammalian” is meant to include human and other primate species, as well as porcine, bovine, canine, murine, and the like.

The term “pancreatic cells” or “cells of pancreatic origin” refers to cells or a population or preparation of cells of pancreatic tissues, including both endocrine and exocrine tissues, as well as cell lines derived therefrom.

The endocrine pancreas is composed of hormone-producing cells arranged in clusters known as islets of Langerhans. Of the four main types of cells that form the islets (“islet cells”), the alpha cells produce glucagons, the beta cells produce insulin, the delta cells produce somatostatin, and the PP cells produce pancreatic polypeptide (PP).

The exocrine pancreas includes the pancreatic acini and the pancreatic duct. Pancreatic acinar cells synthesize a range of digestive enzymes. Ductal cells secrete bicarbonate ions and water in response to the hormone secreted from the gastrointestinal tract.

Thus, “pancreatic cells” or “cells of pancreatic origin”, as used herein, refer to cells found in a mammalian pancreas, including alpha cells, beta cells, delta cells, PP cells, acinar cells, ductal cells or other cells (e.g., endothelial cells, neuronal cells, and progenitor cells that are not differentiated or not fully differentiated or yet to be differentiated), or a mixture or combination thereof, as well as cell lines established from cells of a mammalian pancreas.

Markers characteristic of pancreatic cells include the expression of cell surface proteins or the encoding genes, the expression of intracellular proteins or the encoding genes, cell morphological characteristics, and the production of secretory products such as glucagon, insulin and somatostatin. Those skilled in the art will recognize that known immunofluorescent, immunochemical, polymerase chain reaction, in situ hybridization, Northern blot analysis, chemical or radiochemical or biological methods can readily ascertain the presence or absence of islet cell specific characteristics.

In one embodiment of the present invention, the pancreatic cells employed in the instant methods are obtained from an adult mammalian pancreas. As used herein, the term “adult” refers to a live mammal of any age. Thus, “adult tissues and cells”, as used herein, are distinct from embryonic and fetal tissues and cells.

Pancreatic cells suitable for use in the methods of the present invention may be prepared from a pancreas according to methods well known to those skilled in the art. For example, the harvested pancreas may be incubated with an enzyme solution at or about 37° C. to digest the pancreatic tissue into small clusters of tissue and cells. Following the appropriate digestion time the tissue digest may be filtered to remove large undigested tissue. The digested tissue may then be applied to a density gradient such as Ficoll, polysucrose, dextran, and the like. The density gradient may either be continuous or discontinuous. The tissue loaded density gradient may then be centrifuged, and the cells contained within the digest migrate within the gradient according to their density. The cells are then retrieved from the gradient, washed, and placed in culture. Pancreatic cells prepared in this manner may contain multiple cell types. If desired, the type(s) of cells in a population of pancreatic cells may be determined using techniques that are well known in the art. For example, the use of cell-type specific stains, such as, for example dithizone, that is specific for islet cells. Alternatively, one may perform immunofluorescence staining using antibodies directed to various pancreatic cell specific proteins, such as, for example, insulin, somatostatin, glucagon, pancreatic polypeptide cytokeratins, amylase, and lipase. In addition, a cell type may be determined by its morphology using techniques such as, for example, light microscopy, or electron microscopy.

In one embodiment of the present invention, a population or preparation of pancreatic cells, composed primarily of cells from pancreatic endocrine tissues, is employed in the methods of the present invention. Cells from pancreatic endocrine tissues may be isolated following essentially the same methods described above.

In another embodiment of the present invention, a population or preparation of pancreatic cells, composed primarily of cells from pancreatic exocrine tissues, e.g., pancreatic acinar and duct cells, is employed in the methods of the present invention. Cells from pancreatic exocrine tissues may be isolated following essentially the same methods described above.

By “undifferentiated pancreatic cells” is meant to include pancreatic cells that have undergone some degree of dedifferentiation (“dedifferentiated cells”), and cells present in pancreatic tissues that are yet to be differentiated or have not fully differentiated. Dedifferentiated pancreatic cells are generally characterized by the loss of expression of at least one marker characteristic of fully differentiated pancreatic cells. Cells present in pancreatic tissues that are yet to be differentiated or have not fully differentiated are characterized by the lack of expression of at least one marker characteristic of fully differentiated pancreatic cells. Undifferentiated pancreatic cells also include cell lines that are established from cells obtained from pancreatic tissue and are characterized by the lack of expression of at least one marker characteristic of differentiated pancreatic cells. Markers specific to differentiated pancreatic cells include, but are not limited to, insulin, glucagon, somatostatin, pancreatic polypeptide, amylase, lipase, cytokeratin, PDX-1(pancreatic and duodenal homeobox gene-1), NGN-3 (neurogenin-3), Hlxb9, Nkx6.1, Nkx2.2, MafA, Isl1, NeuroD, HNF1α & β, HNF4α, HNF6, Pax4, Pax6, among others.

As described hereinabove, standard culture conditions, such as oxygen and carbon dioxide concentrations, concentrations of nutrients such as, cell density, temperature, pH, mechanical stress and culture substrates, may be manipulated to promote the dedifferentiation of pancreatic cells to less differentiated cells. For example, nutrient deprivation (e.g., low or no serum, high cell density) may promote dedifferentiation of pancreatic cells, or dedifferentiation of at least some cells in a pancreatic cell population.

Further in accordance with the present invention, a “mesenchymal stem cell” (“MSC”) refers to a cell originated from the mesoderm of a mammal that is not fully differentiated and has the potential to differentiate into a variety of cells or tissues, including: connective tissue, bone, and cartilage, muscle, blood and blood vessels, lymphatic and lymphoid organs, notochord, pleura, pericardium, kidney, and gonads. A mesenchymal stem cell is generally characterized by the expression of at least one of the following surface markers: SH2, SH3, CD29, CD44, CD49, CD71, CD90, CD105, CD106, CD120a, CD124, STOR-1, or other surface proteins, and the lack of expression of CD34, CD45 or Vimentin.

Mesenchymal stem cells suitable for use in the methods of the present invention may be obtained from tissues such as, for example, but not limited to, bone marrow, umbilical cord blood, amniotic sac and fluid, placenta, skin, fat, muscle, vasculature, liver, pancreas, or peripheral blood, using methods that are well known in the art and are further illustrated in the examples described herein below.

By “cultured in the presence of” or “co-culture” is meant that at least two (i.e., two or more) types of cells are physically mixed together and are put in close proximity or contact of each other; or alternatively, the different types of cells are physically separated from each other but share a common medium which allow for the interactions of soluble factors between the different cell types (e.g., by using a cell culture insert). A co-culture may be achieved, for example, by seeding a mixture of different cell types as a heterogeneous population of cells onto a suitable culture substrate. Alternatively, mesenchymal stem cells may first be grown to confluence, which may then serve as a substrate for the second desired cell type to be cultured within the conditioned medium.

By “culture substrate” is meant the environment or base on which the cells live, feed, and grow, such as petri dish, culture flask, bottle, cellular matrix, and the like.

By “conditioned medium” is meant that a population of cells is grown in a medium and contributes soluble factors to the medium. In one such use, the cells are removed from the medium however the soluble factors produced by these cells remain. This medium is then used to nourish a different population of cells in the presence of the soluble factors produced by the initial population of cells.

The cells are co-cultured in basic defined cell culture media, which may be supplemented with serum, serum substitutes or no serum, and with growth factors hormones, cytokines, extracellular matrix components, and media components that may be appropriate.

By “basic defined cell culture medium” is meant a serum reducing, serum free or serum containing, chemically defined cell growth medium. Such medium includes, but is not limited to, Dulbecco's Modified Eagle's Medium (DMEM), alpha modified Minimum Essential Medium (alpha MMEM), Basal Medium Essential (BME), CMRL-1066, RPMI 1640, M199 medium, Ham's F10 nutrient medium or DMEM/F12. These and other useful media are available from GIBCO, Grand Island, N.Y., U.S.A., for example. A number of these media are reviewed in Methods in Enzymology, Volume LVIII, “Cell Culture”, pp. 62-72, edited by William B. Jakoby and Ira H. Pastan, published by Acedemic Press Inc.

Examples of growth factors, hormone, chemokines and cytokines suitable for use in the medium for promoting the growth and proliferation of undifferentiated pancreatic cells may include, but are not limited to, the Fibroblast Growth Factor family of proteins (FGF1-23) including, but not limited to, FGF basic (146 aa), FGF basic (157 aa), FGF acidic, the TGF beta family of proteins including, but not limited to, TGF-beta 1, TGF-beta 2, TGF-beta 3, TGF-beta sRII, Latent TGF-beta, the Tumor necrosis factor (TNF) superfamily (TNFSF) including, but not limited to, TNFSF1-18, including TNF-alpha, TNF-beta, basic fibroblast growth factor (bFGF), transforming growth factor, platelet derived growth factor, vascular endothelial growth factor (VEGF), epidermal growth factor (EGF), leukemia inhibitory factor, steel factor, hepatocyte growth factor (HGF), insulin, erythropoietin, and colony stimulating growth factor CSF, GM-CSF, CCFF, interferons, interleukins, tumor necrosis factors (alpha or beta), bone morphogenic proteins (BMP-2, -4, 6, -7, -11, -12, and -13), fibroblast growth factors-1 and -2 (FGFI and -2), platelet-derived growth factor-AA, and -BB, platelet rich plasma, insulin growth factor (IGF-I, II) growth differentiation factor (GDF-5, -6, -8, -10), glucagon like peptide-I and II (GLP-I and II), Exendin-4, glucose-dependent insulinotropic polypeptide (Gastric Inhibitory Polypeptide, GIP), Ghrelin, retinoic acid, parathyroid hormone, gastrin I and II, estrogen, progesterone, glucocorticoids such as dexamethasone, copper chelators such as triethylene pentamine, TGF-β, TGFα, forskolin, Na-Butyrate, activin, betacellulin, insulin/transferrin/selenium (ITS), keratinocyte growth factor (KGF), islet neogenesis-associated protein (INGAP), Reg protein, the insulin-like growth factor family including, but not limited to, IGF-1 or their binding proteins including, but not limited to, IGFBP-1, II-1 R rp2, IGFBP-5, IGFBP-6, the matrix metalloproteinases including, but not limited to, MMP-1, CF, MMP-2, CF, MMP-2 (NSA-expressed), CF, MMP-7, MMp-8, MMP-10, MMP-9, TIMP-1, CF, TIMP-2, PDGF, Flt-3 ligand, B7-1(CD80), B7-2(CD86), DR6, IL-13 R alpha, IL-15 R alpha, GRO beta/CXCL2 (aa 39-107), IL 1-18, II-8/CXCL8, GDNF, G-CSF, GM-CSF, M-GSF, PDGF-BB, PDGF-AA, PDGF-AB, IL-2 sR alpha, IL-2 sR beta, Soluble TNF RII, IL-6 sR, Soluble gp130, PD-ECGF, IL-4 sR, beta-ECGF, TGF-alpha, TGF-beta sRII, TGF-beta 5, LAP (TGF-beta 1), BDNF, LIF sR alpha, LIF, KGF/FGF-7, Pleiotrophin, ENA-78/CXCL5, SCF, beta-NGF, CNTF, Midkine, HB-EGF, SLPI, Betacellulin, Amphiregulin, PIGF, Angiogenin, IP-10ICXCL10, NT-3, NT-4, MIP-1 alpha/CCL3, MIP-1 beta/CCL4, I-309/CCL1, GRO alpha/CXCL1, GRO beta/CXCL2, GRO gamma/CXCL3, Rantes/CCL5, MCP-1/CCL2, MCP-2/CCL8, MCP- 3/CCL7, IFN-gamma, Erythropoietin, Thrombopoietin, MIF, IGF-I, IGF-II, VEGF, HGF, Oncostatin M, HRG-alpha (EGF Domain), TGF-beta 2, CNTF R alpha, Tie-2/Fc Chimera, BMP-4, BMPR-IA, Eotaxin/CCL11, VEGF R1 (Fit-1), PDGF sR alpha, HCC-1/CCL 14, CTLA-4, MCP-4/CCL13, GCP-2/CXCL6, TECK/CCL25, MDC/CCL22, Activin A, Eotaxin-2/MPIF-2/CCL24, Eotaxin-3/CCL-26 (aa 24-94), TRAIL R1 (DR4), VEGF R3 (Fit-4)/SDF-1 alpha(PBSF)/CXCL12, MSP, BMP-2, HVEM/VEGF R2 (KDR), Ephrin-A3, MIP-3 alpha/CCL20, MIP-3 beta/CCL19, Fractalkine/CX3CL1 (Chemokine Domain), TARC/CCL17, 6Ckine/CCL21, p75 Neurotrophin R (NGF R), SMDF, Neurturin, Leptin R/Fc Chimera, MIG/CXCL9, NAP-2/CXCL7, PARC/CCL18, Cardiotrophin-1 (CT-1), GFR alpha-2, BMP-5, IL-8/CXCL8 (Endothelial Cell Derived), Tie-1, Viral CMV UL146, VEGF-D, Angiopoietin-2, Inhibin A, TRANCE/RANK L, CD6/Fc Chimera, CF, dMIP-1 delta/LKN-1/CCL15(68 aa), TRAIL R3/Fc Chimera, Soluble TNF RI, Activin RIA, EphA1, E-Cadherin, ENA-70, ENA-74, Eotaxin-3/CCL26, ALCAM, FGFR1 alpha (IIIc), Activin B, FGFT1 beta (IIIc), LIGHT, FGFR2 beta(IIIb), DNAM-1, Follistatin, GFR alpha-3, gp 130, I-TAC/CXCL11, IFN-gamma RI, IGFBP-2, IGFBP-3, Inhibin B, Prolactin CF, RANK, FGFR2 beta (IIIc), FGFR4, TrkB, GITR, MSP R, GITR Ligand, Lymphotactin/XCL1, FGFR2 alpha (IIIc), Activin AB, ICAM-3 (CD50), ICAM-1(CD54), TNF RII, L-Selectin (CD62L, BLC/BCA-1/CXCL13, HCC-4/CCL16, ICAM-2(CD102), IGFBP-4, Osteoprotegerin)OPG), uPAR, Activin RIB, VCAM-1 (CD106), CF, BMPR-II, IL-18 R, IL-12 R beta 1, Dtk, LBP, SDF-I alpha (PBSF)/CXCL12 (synthetic), E-Selectin (CD62E), L-Selectin (CD62L), P-Selectin (CD62P), ICAM-1 (CD54), VCAM-1 (CD106), CD31 (PECAM-1),hedgehog family of proteins, Interleukin-10, Heregulin, HER4, Heparin Binding Epidermal Growth Factor, NGF (Nerve growth factor), MIP-18, MIP-2, MCP-1, MCP-5, NGF, NGF-B, leptin, Interferon A, Interferon A/D, Interferon B, Interferon Inducible Protein-10, Insulin Like Growth Factor-II, IGBFBP/IGF-1 Complex, C10, Cytokine Induced Neutrophil Chemoattractant 2, Cytokine Induced Neutrophil Chemoattractant 2B, Cytokine Induced Neutrophil Chemoattractant 1, Cytokine Responsive Gene-2, or any fragment thereof, or their neutralizing antibodies.

Factors involved in cell-cell interactions that may be included in the culture medium include, but are not limited to, the ADAM (A Disintegrin and Metalloproteinase) family of proteins including ADAM 1,2,3A, 3B, 4-31 and TS1-9, ADAMTSs (ADAMs with thrombospondin motifs), Reprolysins, metzincins, zincins, zinc metalloproteinases or their neutralizing antibodies.

Additional components that may be included in the culture medium include natural or synthetic compounds or peptides that effect differentiation or signaling pathways including kinases, for example JAK, MAP, Jun kinase (JNK), p38, Akt, PKC, Calmodulin, Tyrosine kinase, SMAD, ERK, MEK, ErbB, FAK, PI3K, proteasome, ion channel blocker, or adhesion molecules including, but not limited to, Ig superfamily CAM'S, Integrins, Cadherins, Selectins or their neutralizing antibodies.

Furthermore, the culture medium may include nucleic acids that encode or block (via antisense, ribozyme activity, or RNA interference, transcription factors, siRNA, RNAi that are involved in regulating gene expression during differentiation) genes coding for growth factors, cytokines, extracellular matrix components, or other molecules that regulate differentiation.

Suitable extracellular matrix components that may be included in the culture medium include, but are not limited to, Keratin Sulphate Proteoglycan, Laminin, Chondroitin Sulphate A, SPARC, beta amyloid precursor protein, beta amyloid, presenilin 1,2, apolipoprotein E, thrombospondin-1,2, Heparan Sulphate, Heparan sulphate proteoglycan, Matrigel, Aggregan, Biglycan, Poly-L-Ornithine, the collagen family including but not limited to Collagen I-IV, Poly-D-Lysine, Ecistatin (Viper Venom), Flavoridin (Viper Venom), Kistrin (Viper Venom), Vitronectin, Superfibronectin, Fibronectin Adhesion-Promoting peptide, Fibronectin Fragment III-C, Fibronectin Fragment-30 KDA, Fibronectin-Like Polymer, Fibronectin Fragment 45 KDA, Fibronectin Fragment 70 KDA, Asialoganglioside-GM, Disialoganglioside-GOLA, Monosialo Ganglioside-GM₁, Monosialoganglioside-GM₂, Monosialoganglioside-GM₃, Methylcellulose, Keratin Sulphate Proteoglycam, Laminin or Chondroitin Sulphate A.

Other media components that may be included in the culture medium include, but are not limited to, glucose, lipids, transferrin, ITS (insulin, transferrin and Selenium), Nicotinamide, 2-Mercaptoethanol, B-Cyclodextrin, Prostaglandin F2, Somatostatin Thyrotropin Releasing Hormone, L-Thyroxine, 3,3,5-Triiodo-L-Thyronine, L-Ascorbic Acid, Fetuin, Heparin, 2-Mercaptoethanol, Horse Serum, DMSO, Chicken Serum, Goat Serum, Rabbit Serum, Human Serum, Pituitary Extract, Stromal Cell Factor, Conditioned Medium, Hybridoma Medium, d-Aldosterone, Dexamethasone, DHT, B-Estradiol, Glucagon, Insulin, Progesterone, Prostaglandin-D₂, Prostaglandin-E₁, Prostaglandin-E₂, Prostaglandin-F₂, Serum-Free Medium, Endothelial Cell Growth Supplement, Gene Therapy Medium, MDBK-GM Medium, QBSF-S1, Endothelial Medium, Keratinocyte Medium, Melanocyte Medium, Gly-His-Lys, soluble factors that inhibit or interfere with intracellular enzymes or other molecules including, but not limited to, compounds that alter chromatin modifying enzymes such as histone deacetylases, butyrate or trichostatin A, compounds that modulates cAMP, protein kinase inhibitors, compounds that alter intracellular calcium concentration, or compounds that modulate phosphatidylinositol signaling pathways.

According to the present invention, insulin-producing cells are generated or expanded as a result of the co-culture of pancreatic cells and mesenchymal stem cells.

According to the present invention, precursors of insulin-producing cells are generated or expanded as a result of the co-culture of undifferentiated pancreatic cells and mesenchymal stem cells. Accordingly, another embodiment of the present invention is directed to a method of promoting the generation and/or expansion of precursors of insulin-producing cells by culturing undifferentiated pancreatic cells or a population of pancreatic cells in the presence of mesenchymal stem cells.

Without intending to be bound by any particular theory, it is proposed that pancreatic cells, when cultured in the presence of mesenchymal stem cells, may proliferate and develop into insulin-producing cells or precursors thereof. Thus, mesenchymal stem cells may also promote the proliferation and growth of precursors of insulin-producing cells that are present in a pancreatic cell preparation. Additionally, mesenchymal stem cells, when co-cultured with pancreatic cells, can themselves differentiate or transdifferentiate into precursors of insulin-producing cells.

By “transdifferentiate” or “transdifferentiation” is meant a non-stem cell whichtransforms into a different type of cell, or, alternatively, a stem cell that has already differentiated with certain specialization, e.g., a mesenchymal stem cell (which normally gives rise to connective tissue, bone, and cartilage, muscle, blood and blood vessels, lymphatic and lymphoid organs, notochord, pleura, pericardium, kidney, and gonads), develops or differentiates outside its already established differentiation., e.g., a mesenchymal stem cell gives rise to a pancreatic cell or insulin-producing cell.

By “precursors of insulin-producing cells” refer to precursor cells that are not fully differentiated but have the potential to further differentiate into insulin-producing cells. Such precursor cells are characterized by the lack of expression of at least one marker specific to fully differentiated pancreatic cells. The precursor cells can express one or more of beta cell lineage specific markers including, but not limited to, the expression of transcription factors such as PDX-1 (pancreatic and duodenal homeobox gene-1), NGN-3(neurogenin-3), Hlxb9, Nkx6.1, Nkx2.2, MafA, Isl1, NeuroD, HNF1α & β, HNF4 α, HNF6, Pax4, Pax6 and others. These transcription factors are well established in prior art for identification of endocrine cells (Development, Vol. 131, No. 1, 165-179, 2004).

By “beta cell lineage” is meant the ancestry of pancreatic beta islet cells, including ancestral cells and all of the subsequent cell divisions which occurred to produce the beta islet cells.

Mesenchymal stem cells and undifferentiated pancreatic cells may be co-cultured in basic defined cell culture media, which may be supplemented with serum, serum substitutes or no serum, and with growth factors hormones, cytokines, extracellular matrix components, and media components that may be appropriate.

Examples of growth factors, hormone, chemokines and cytokines suitable for use in the medium for promoting the generation and proliferation of precursors of insulin-producing cells may include, but are not limited to, the Fibroblast Growth Factor family of proteins (FGF1-23) including, but not limited to, FGF basic (146 aa), FGF basic (157 aa), FGF acidic, the TGF beta family of proteins including, but not limited to, TGF-beta 1, TGF-beta 2, TGF-beta 3, TGF-beta sRII, Latent TGF-beta, the Tumor necrosis factor (TNF) superfamily (TNFSF) including, but not limited to, TNFSF1-18, including TNF-alpha, TNF-beta, basic fibroblast growth factor (bFGF), transforming growth factor, platelet derived growth factor, vascular endothelial growth factor (VEGF), epidermal growth factor (EGF), leukemia inhibitory factor, steel factor, hepatocyte growth factor (HGF), insulin, erythropoietin, and colony stimulating growth factor CSF, GM-CSF, CCFF, interferons, interleukins, tumor necrosis factors (alpha or beta), bone morphogenic proteins (BMP-2, -4, 6, -7,-11, -12, and -13), fibroblast growth factors-1 and-2 (FGF1 and-2), platelet-derived growth factor-AA, and-BB, platelet rich plasma, insulin growth factor (IGF-I, II) growth differentiation factor (GDF-5, -6, -8, -10), glucagon like peptide-I and II (GLP-I and II), Exendin-4, glucose-dependent insulinotropic polypeptide (Gastric Inhibitory Polypeptide, GIP), Ghrelin, retinoic acid, parathyroid hormone, gastrin I and II, estrogen, progesterone, glucocorticoids such as dexamethasone, copper chelators such as triethylene pentamine, TGF-β, TGFα, forskolin, Na-Butyrate, activin, betacellulin, insulin/transferrin/selenium (ITS), keratinocyte growth factor (KGF), islet neogenesis-associated protein (INGAP), Reg protein, the insulin-like growth factor family including, but not limited to, IGF-1 or their binding proteins including, but not limited to, IGFBP-1, II-1 R rp2, IGFBP-5, IGFBP-6, the matrix metalloproteinases including, but not limited to, MMP-1, CF, MMP-2, CF, MMP-2 (NSA-expressed), CF, MMP-7, MMp-8, MMP-10, MMP-9, TIMP-1, CF, TIMP-2, PDGF, Flt-3 ligand, B7-1(CD80), B7-2(CD86), DR6, IL-13 R alpha, IL-15 R alpha, GRO beta/CXCL2 (aa 39-107), IL 1-18, II-8/CXCL8, GDN G-CSF, GM-CSF, M-GSF, PDGF-BB, PDGF-AA, PDGF-AB, IL-2 sR alpha, IL-2 sR beta, Soluble TNF RII, IL-6 sR, Soluble gp130, PD-ECGF, IL-4 sR, beta-ECGF, TGF-alpha, TGF-beta sRII, TGF-beta 5, LAP (TGF-beta 1), BDNF, LIF sR alpha, LIF, KGF/FGF-7, Pleiotrophin, ENA-78/CXCL5, SCF, beta-NGF, CNTF, Midkine, HB-EGF, SLPI, Betacellulin, Amphiregulin, PIGF, Angiogenin, IP-10ICXCL10, NT-3, NT-4,MIP-1 alpha/CCL3, MIP-1 beta/CCL4, I-309/CCL1, GRO alpha/CXCL1, GRO beta/CXCL2, GRO gamma/CXCL3, Rantes/CCL5, MCP-1/CCL2, MCP-2/CCL8, MCP-3/CCL7, IFN-gamma, Erythropoietin, Thrombopoietin, MIF, IGF-I, IGF-II, VEGF, HGF, Oncostatin M, HRG-alpha (EGF Domain), TGF-beta 2, CNTF R alpha, Tie-2/Fc Chimera, BMP-4, BMPR-IA, Eotaxin/CCL11, VEGF R1 (Fit-1), PDGF sR alpha, HCC-1/CCL 14, CTLA-4, MCP-4/CCL13, GCP-2/CXCL6, TECK/CCL25, MDC/CCL22, Activin A, Eotaxin-2/MPIF-2/CCL24, Eotaxin-3/CCL-26 (aa 24-94), TRAIL R1 (DR4), VEGF R3 (Fit-4) /SDF-1 alpha(PBSF)/CXCL12, MSP, BMP-2, HVEM/VEGF R2 (KDR), Ephrin-A3, MIP-3 alpha/CCL20, MIP-3 beta/CCL19, Fractalkine/CX3CL1 (Chemokine Domain), TARC/CCL17, 6Ckine/CCL21, p75 Neurotrophin R (NGF R), SMDF, Neurturin, Leptin R/Fc Chimera, MIG/CXCL9, NAP-2/CXCL7, PARC/CCL18, Cardiotrophin-1 (CT-1), GFR alpha-2, BMP-5, IL-8/CXCL8 (Endothelial Cell Derived), Tie-1, Viral CMV UL146, VEGF-D, Angiopoietin-2, Inhibin A, TRANCE/RANK L, CD6/Fc Chimera, CF, dMIP-1 delta/LKN-1/CCL15(68 aa), TRAIL R3/Fc Chimera, Soluble TNF RI, Activin RIA, EphA1, E-Cadherin, ENA-70, ENA-74, Eotaxin-3/CCL26, ALCAM, FGFR1 alpha (IIIc), Activin B, FGFT1 beta (IIIc), LIGHT, FGFR2 beta(IIIb), DNAM-1, Follistatin, GFR alpha-3, gp 130, I-TAC/CXCL11, IFN-gamma RI, IGFBP-2, IGFBP-3, Inhibin B, Prolactin CF, RANK, FGFR2 beta (IIIc), FGFR4, TrkB, GITR, MSP R, GITR Ligand, Lymphotactin/XCL1, FGFR2 alpha (IIIc), Activin AB, ICAM-3 (CD50), ICAM-1(CD54), TNF RII, L-Selectin (CD62L, BLC/BCA-1/CXCL13, HCC-4/CCL16, ICAM-2(CD102), IGFBP-4, Osteoprotegerin)OPG), uPAR, Activin RIB, VCAM-1 (CD106), CF, BMPR-II, IL-18 R, IL-12 R beta 1, Dtk, LBP, SDF-I alpha (PBSF)/CXCL12 (synthetic), E-Selectin (CD62E), L-Selectin (CD62L), P-Selectin (CD62P), ICAM-1 (CD54), VCAM-1 (CD106), CD31 (PECAM-1),hedgehog family of proteins, Interleukin-10, Heregulin, HER4, Heparin Binding Epidermal Growth Factor, NGF (Nerve growth factor), MIP-18, MIP-2, MCP-1, MCP-5, NGF, NGF-B, leptin, Interferon A, Interferon A/D, Interferon B, Interferon Inducible Protein-10, Insulin Like Growth Factor-II, IGBFBP/IGF-1 Complex, C10, Cytokine Induced Neutrophil Chemoattractant 2, Cytokine Induced Neutrophil Chemoattractant 2B, Cytokine Induced Neutrophil Chemoattractant 1, Cytokine Responsive Gene-2, or any fragment thereof or their neutralizing antibodies.

Factors involved in cell-cell interactions that may be included in the culture medium include, but are not limited to, the ADAM (A Disintegrin and Metalloproteinase) family of proteins including ADAM 1,2,3A, 3B, 4-31 and TS 1-9, ADAMTSs (ADAMs with thrombospondin motifs), Reprolysins, metzincins, zincins, zinc Metalloproteinases, or their neutralizing antibodies.

Additional components that may be included in the culture medium include natural or synthetic compounds or peptides that effect differentiation or signaling pathways including kinases for example JAK, MAP, Jun kinase (JNK), p38, Akt, PKC, Calmodulin, Tyrosine kinase, SMAD, ERK, MEK, ErbB, FAK, P13K, proteasome, ion channel blocker, and adhesion molecules including, but not limited to, Ig superfamily CAM's, Integrins, Cadherins, Selectins, or their neutralizing antibodies.

Furthermore, the culture medium may include nucleic acids that encode or block (via antisense, ribozyme activity, or RNA interference, transcription factors, siRNA, RNAi that are involved in regulating gene expression during differentiation) genes coding for growth factors, cytokines, extracellular matrix components, or other molecules that regulate differentiation.

Suitable extracellular matrix components that may be included in the culture medium include, but are not limited to, Keratin Sulphate Proteoglycan, Laminin, Chondroitin Sulphate A, SPARC, beta amyloid precursor protein, beta amyloid, presenilin 1,2, apolipoprotein E, thrombospondin-1,2, Heparan Sulphate, Heparan sulphate proteoglycan, Matrigel, Aggregan, Biglycan, Poly-L-Ornithine, the collagen family including but not limited to Collagen I-IV, Poly-D-Lysine, Ecistatin (Viper Venom), Flavoridin (Viper Venom), Kistrin (Viper Venom), Vitronectin, Superfibronectin, Fibronectin Adhesion-Promoting peptide, Fibronectin Fragment III-C, Fibronectin Fragment-30 KDA, Fibronectin-Like Polymer, Fibronectin Fragment 45 KDA, Fibronectin Fragment 70 KDA, Asialoganglioside-GM, Disialoganglioside-GOLA, Monosialo Ganglioside-GM₁, Monosialoganglioside-GM₂, Monosialoganglioside-GM₃, Methylcellulose, Keratin Sulphate Proteoglycam, Laminin and Chondroitin Sulphate A.

Other media components that may be included in the culture medium include, but are not limited to, glucose, lipids, transferrin, ITS (insulin, transferrin and Selenium), Nicotinamide, 2-Mercaptoethanol, B-Cyclodextrin, Prostaglandin F₂, Somatostatin Thyrotropin Releasing Hormone, L-Thyroxine, 3,3,5-Triiodo-L-Thyronine, L-Ascorbic Acid, Fetuin, Heparin, 2-Mercaptoethanol, Horse Serum, DMSO, Chicken Serum, Goat Serum, Rabbit Serum, Human Serum, Pituitary Extract, Stromal Cell Factor, Conditioned Medium, Hybridoma Medium, d-Aldosterone, Dexamethasone, DHT, B-Estradiol, Glucagon, Insulin, Progesterone, Prostaglandin-D₂, Prostaglandin-E₁, Prostaglandin-E₂, Prostaglandin-F₂, Serum-Free Medium, Endothelial Cell Growth Supplement, Gene Therapy Medium, MDBK-GM Medium, QBSF-S1, Endothelial Medium, Keratinocyte Medium, Melanocyte Medium, Gly-His-Lys, soluble factors that inhibit or interfere with intracellular enzymes or other molecules including, but not limited to, compounds that alter chromatin modifying enzymes such as histone deacetylases, butyrate or trichostatin A, compounds that modulates cAMP, protein kinase inhibitors, compounds that alter intracellular calcium concentration, or compounds that modulate phosphatidylinositol signaling pathways.

Mesenchymal stem cells may also be cultured in a conditioned medium from a culture of pancreatic cells, and induced to differentiate or transdifferentiate into precursors of insulin-producing cells and ultimately insulin-producing cells. The conditioned medium may provide cellular factors such as cytokines, growth factors, hormones, and extracellular matrix. For example, a medium from a culture of islet cells or injured islet cells, or a culture of islets from new born, or a culture of regenerating islet tissue or cell lysates, can be used to culture MSCs to differentiate or transdifferentiate MSCs into precursors of insulin-producing cells and ultimately insulin-producing cells.

In still another embodiment, the present invention provides a method of generating insulin-producing cells by culturing undifferentiated pancreatic cells, or a population of pancreatic cells containing undifferentiated pancreatic cells, in the presence of mesenchymal stem cells to generate and/or expand precursors of insulin-producing cells, and further developing the precursors into mature insulin-producing cells.

According to the present invention, the precursors of insulin-producing cells that are produced as a result of a co-culture of undifferentiated pancreatic cells and mesenchymal stem cells, as described hereinabove, may develop into mature insulin-producing cells either in vitro or in vivo.

For further differentiation in vitro towards mature insulin-producing cells, precursors of insulin-producing cells may be cultured in a basic defined medium such as DMEM, supplemented with components that support cell growth such as bovine serum albumin (BSA), Human Serum Albumin (HSA), Fetal Bovine Serum (FCS), Newborn Calf Serum (NCS), Equine Serum (ES), Human Serum (HS), penicillin/streptomycin (P/S) or nicotinamide, or with one or more growth factors, hormones, cytokines, extracellular matrix components, or media components.

Examples of growth factors, hormone, chemokines and cytokines suitable for use in the medium for promoting the further differentiation of precursors of insulin-producing cells may include, but are not limited to, the Fibroblast Growth Factor family of proteins (FGF1-23) including but not limited to FGF basic (146 aa), FGF basic (157 aa), FGF acidic, the TGF beta family of proteins including but not limited to TGF-beta 1, TGF-beta 2, TGF-beta 3, TGF-beta sRII, Latent TGF-beta, the Tumor necrosis factor (TNF) superfamily (TNFSF) including but not limited to TNFSF1-18, including TNF-alpha, TNF-beta, basic fibroblast growth factor (bFGF), transforming growth factor, platelet derived growth factor, vascular endothelial growth factor (VEGF), epidermal growth factor (EGF), leukemia inhibitory factor, steel factor, hepatocyte growth factor (HGF), insulin, erythropoietin, and colony stimulating growth factor CSF, GM-CSF, CCFF, interferons, interleukins, tumor necrosis factors (alpha or beta), bone morphogenic proteins (BMP-2, -4, 6, -7, -11, -12, and -13), fibroblast growth factors-1 and-2 (FGF1and-2), platelet-derived growth factor-AA, and-BB, platelet rich plasma, insulin growth factor (IGF-I, II) growth differentiation factor (GDF-5, -6, -8, -10), glucagon like peptide-I and II (GLP-I and II), Exendin-4, glucose-dependent insulinotropic polypeptide (Gastric Inhibitory Polypeptide, GIP), Ghrelin, retinoic acid, parathyroid hormone, gastrin I and II, estrogen, progesterone, glucocorticoids such as dexamethasone, copper chelators such as triethylene pentamine, TGF-β, TGFα, forskolin, Na-Butyrate, activin, betacellulin, insulin/transferrin/selenium (ITS), keratinocyte growth factor (KGF), islet neogenesis-associated protein (INGAP), Reg protein, the insulin-like growth factor family including but not limited to IGF-1 or their binding proteins including but not limited to IGFBP-1, II-1 R rp2, IGFBP-5, IGFBP-6, the matrix metalloproteinases including but not limited to MMP-1, CF, MMP-2, CF, MMP-2 (NSA-expressed), CF, MMP-7, MMp-8, MMP-10, MMP-9, TIMP-1, CF, TIMP-2, PDGF, Flt-3 ligand, B7-1(CD80), B7-2(CD86), DR6 , IL-13 R alpha, IL-15 R alpha, GRO beta/CXCL2 (aa 39-107), IL 1-18, II-8/CXCL8, GDNF, G-CSF, GM-CSF, M-GSF, PDGF-BB, PDGF-AA, PDGF-AB, IL-2 sR alpha, IL-2 sR beta, Soluble TNF RII, IL-6 sR, Soluble gp130, PD-ECGF, IL-4 sR, beta-ECGF, TGF-alpha, TGF-beta sRII, TGF-beta 5, LAP (TGF-beta 1), BDNF, LIF sR alpha, LIF, KGF/FGF-7, Pleiotrophin, ENA-78/CXCL5, SCF, beta-NGF, CNTF, Midkine, HB-EGF, SLPI, Betacellulin, Amphiregulin, PIGF, Angiogenin, IP-10ICXCL10, NT-3, NT-4, MIP-1 alpha/CCL3, MIP-1 beta/CCL4, I-309/CCL1, GRO alpha/CXCL1, GRO beta/CXCL2, GRO gamma/CXCL3, Rantes/CCL5, MCP-1/CCL2, MCP-2/CCL8, MCP-3/CCL7, IFN-gamma, Erythropoietin, Thrombopoietin, MIF, IGF-I, IGF-II, VEGF, HGF, Oncostatin M, HRG-alpha (EGF Domain), TGF-beta 2, CNTF R alpha, Tie-2/Fc Chimera, BMP-4, BMPR-IA, Eotaxin/CCL11, VEGF R1 (Fit-1), PDGF sR alpha, HCC-1/CCL 14, CTLA-4, MCP-4/CCL13, GCP-2/CXCL6, TECK/CCL25, MDC/CCL22, Activin A, Eotaxin-2/MPIF-2/CCL24, Eotaxin-3/CCL-26 (aa 24-94), TRAIL RI (DR4), VEGF R3 (Fit-4) /SDF-1 alpha(PBSF)/CXCL12, MSP, BMP-2, HVEM/VEGF R2 (KDR), Ephrin-A3, MIP-3 alpha/CCL20, MIP-3 beta/CCL19, Fractalkine/CX3CL1 (Chemokine Domain), TARC/CCL17, 6Ckine/CCL21, p75 Neurotrophin R (NGF R), SMDF, Neurturin, Leptin R/Fc Chimera, MIG/CXCL9, NAP-2/CXCL7, PARC/CCL18, Cardiotrophin-1 (CT-1), GFR alpha-2, BMP-5, IL-8/CXCL8 (Endothelial Cell Derived), Tie-1, Viral CMV UL146, VEGF-D, Angiopoietin-2, Inhibin A, TRANCE/RANK L, CD6/Fc Chimera, CF, dMIP-1 delta/LKN-1/CCL15(68 aa), TRAIL R3/Fc Chimera, Soluble TNF R1, Activin RIA, EphA1, E-Cadherin, ENA-70, ENA-74, Eotaxin-3/CCL26, ALCAM, FGFR1 alpha (IIIc), Activin B, FGFT1 beta (IIIc), LIGHT, FGFR2 beta(IIIb), DNAM-1, Follistatin, GFR alpha-3, gp 130, I-TAC/CXCL11, IFN-gamma RI, IGFBP-2, IGFBP-3, Inhibin B, Prolactin CF, RANK, FGFR2 beta (IIIc), FGFR4, TrkB, GITR, MSP R, GITR Ligand, Lymphotactin/XCL1, FGFR2 alpha (IIIc), Activin AB, ICAM-3 (CD50), ICAM-1(CD54), TNF RII, L-Selectin (CD62L, BLC/BCA-1/CXCL13, HCC-4/CCL16, ICAM-2(CD102), IGFBP-4, Osteoprotegerin)OPG), uPAR, Activin RIB, VCAM-1 (CD106), CF, BMPR-II, IL-18 R, IL-12 R beta 1, Dtk, LBP, SDF-I alpha (PBSF)/CXCL12 (synthetic), E-Selectin (CD62E), L-Selectin (CD62L), P-Selectin (CD62P), ICAM-1 (CD54), VCAM-1 (CD106), CD31 (PECAM-1),hedgehog family of proteins, Interleukin-10, Heregulin, HER4, Heparin Binding Epidermal Growth Factor, NGF (Nerve growth factor), MIP-18, MIP-2, MCP-1, MCP-5, NGF, NGF-B, leptin, Interferon A, Interferon A/D, Interferon B, Interferon Inducible Protein-10, Insulin Like Growth Factor-II, IGBFBP/IGF-1 Complex, C10, Cytokine Induced Neutrophil Chemoattractant 2, Cytokine Induced Neutrophil Chemoattractant 2B, Cytokine Induced Neutrophil Chemoattractant 1, Cytokine Responsive Gene-2, or any fragment thereof, or their neutralizing antibodies.

Factors involved in cell-cell interactions that may be included in the culture medium include, but are not limited to, the ADAM (A Disintegrin and Metalloproteinase) family of proteins including ADAM 1,2,3A, 3B, 4-31 and TS1-9, ADAMTSs (ADAMs with thrombospondin motifs), Reprolysins, metzincins, zincins, and zinc Metalloproteinases, or their neutralizing antibodies.

Additional components that may be included in the culture medium include natural or synthetic compounds or peptides that effect differentiation or signaling pathways including kinases for example JAK, MAP, Jun kinase (JNK), p38, Akt, PKC, Calmodulin, Tyrosine kinase, SMAD, ERK, MEK, ErbB, FAK, PI3K, proteasome, ion channel blocker, and adhesion molecules including but not limited to Ig superfamily CAM's, Integrins, Cadherins, Selectins or their neutralizing antibodies.

Furthermore, the culture medium may include nucleic acids that encode or block by antisense, ribozyme activity, or RNA interference, transcription factors, siRNA, RNAi that are involved in regulating gene expression during differentiation, genes coding for growth factors, cytokines, extracellular matrix components, or other molecules that regulate differentiation.

Suitable extracellular matrix components that may be included in the culture medium include but are not limited to Keratin Sulphate Proteoglycan, Laminin, Chondroitin Sulphate A, SPARC, beta amyloid precursor protein, beta amyloid, presenilin 1,2, apolipoprotein E, thrombospondin-1,2, Heparan Sulphate, Heparan sulphate proteoglycan, Matrigel, Aggregan, Biglycan, Poly-L-Ornithine, the collagen family including but not limited to Collagen I-IV, Poly-D-Lysine, Ecistatin (Viper Venom), Flavoridin (Viper Venom), Kistrin (Viper Venom), Vitronectin, Superfibronectin, Fibronectin Adhesion-Promoting peptide, Fibronectin Fragment III-C, Fibronectin Fragment-30 KDA, Fibronectin-Like Polymer, Fibronectin Fragment 45 KDA, Fibronectin Fragment 70 KDA, Asialoganglioside-GM, Disialoganglioside-GOLA, Monosialo Ganglioside-GM₁, Monosialoganglioside-GM₂, Monosialoganglioside-GM₃, Methylcellulose, Keratin Sulphate Proteoglycam, Laminin or Chondroitin Sulphate A.

Other media components that may be included in the culture medium include, but are not limited to, glucose, lipids, transferrin, ITS (insulin, transferrin and Selenium), Nicotinamide, 2-Mercaptoethanol, B-Cyclodextrin, Prostaglandin F₂, Somatostatin Thyrotropin Releasing Hormone, L-Thyroxine, 3,3,5-Triiodo-L-Thyronine, L-Ascorbic Acid, Fetuin, Heparin, 2-Mercaptoethanol, Horse Serum, DMSO, Chicken Serum, Goat Serum, Rabbit Serum, Human Serum, Pituitary Extract, Stromal Cell Factor, Conditioned Medium, Hybridoma Medium, d-Aldosterone, Dexamethasone, DHT, B-Estradiol, Glucagon, Insulin, Progesterone, Prostaglandin-D₂, Prostaglandin-E₁, Prostaglandin-E₂, Prostaglandin-F₂, Serum-Free Medium, Endothelial Cell Growth Supplement, Gene Therapy Medium, MDBK-GM Medium, QBSF-S1, Endothelial Medium, Keratinocyte Medium, Melanocyte Medium, Gly-His-Lys, soluble factors that inhibit or interfere with intracellular enzymes or other molecules including but not limited to compounds that alter chromatin modifying enzymes such as histone deacetylases, butyrate or trichostatin A, compounds that modulates cAMP, protein kinase inhibitors, compounds that alter intracellular calcium concentration, or compounds that modulate phosphatidylinositol signaling pathways.

The growth factors may be included at a concentration that induce or favor the differentiation of the precursor cells toward mature insulin-producing cells over a time period of about one to four weeks.

As described hereinabove, the precursor cells can also further differentiate into insulin-producing cells in an in vivo environment. According to this aspect of the present invention, the precursor cells can be administered (e.g., via implanted or injected) into a suitable mammalian recipient. Prior to administration, the precursor cells may be provided in a biocompatible degradable polymeric scaffold, porous non-degradable device or encapsulation for administration into the recipient.

To enhance further differentiation and survival of implanted cells, additional factors, such as growth factors including those identified hereinabove, antioxidants, anti-inflammatory or angiogenic agents, may be administered. These additional factors may be applied to the recipient mammal, simultaneously with, or after the administration of the precursor cells. For example, the precursor cells and one or more growth factors may be included in the same device or encapsulation for implantation.

In one embodiment, mesenchymal stem cells are also provided to the recipient mammal to facilitate the survival, growth and further differentiation of the precursor cells in vivo. Mesenchymal stem cells may be provided (e.g., by implantation or injection) to the recipient mammal, either separately or together with the precursor of the insulin-producing cells.

Mature insulin-producing cells or tissues can be isolated using methods well known in the art, such as, for example, immunoaffinity purification or FACS. Immunoaffinity purification may be achieved by targeting cell surface molecules expressed by the cells to be purified.

It is believed that mesenchymal stem cells supply the signals that promote the proliferation and differentiation of precursors of insulin-producing cells that are present in the body of the mammal. Alternatively or additionally, the administered mesenchymal stem cells, upon interaction with cells in the pancreas of the mammal, can develop or transdifferentiate into insulin-producing cells.

Mesenchymal stem cells may be administered to the mammal by local injection in the pancreas of the mammal, or by implantation in the pancreas or at a site proximate to the pancreas of the mammal.

The insulin-producing cells as well as the precursors of insulin-producing cells, that are generated in accordance with the present methods described hereinabove, form another embodiment of the present invention.

In another embodiment, the present invention further provides a method of treating a diabetic subject by administering insulin-producing cells or precursors thereof produced in accordance with the methods of the present invention. When precursors of insulin-producing cells are used, such cells may fully differentiate into insulin-producing cells in the subject after transplantation. Mesenchymal stem cells may be administered separately or together with the precursor cells to facilitate the survival and further differentiation of the precursor cells in the subject.

In yet another embodiment, the present invention further provides a method of treating a subject determined to be predisposed to develop diabetes by administering to the subject insulin-producing cells or precursors thereof produced in accordance with the methods of the present invention. When precursors of insulin-producing cells are used, such cells can fully differentiate into insulin-producing cells in the subject after administration (e.g., via transplantation). Mesenchymal stem cells may be administered separately or together with the precursor cells to facilitate the survival and further differentiation of the precursor cells in the subject.

The cells may be used as dispersed cells or formed into clusters that may be infused into the hepatic portal vein. Alternatively, the cells may be provided in biocompatible degradable polymeric scaffolds, porous non-degradable devices or encapsulation for implantation into an appropriate site in the subject, or through any device suitable for cell delivery and/or implantation. The site may be selected from, but not limited to, the liver, the natural pancreas, the renal subcapsular space, the mesentery, the omentum, a subcutaneous pocket, the peritoneum, or other such sites that would ensure cell viability following implantation.

To enhance further differentiation, survival or activity of implanted cells, additional factors, such as growth factors including those identified hereinabove, antioxidants, anti-inflammatory or angiogenic agents, may be administered. These additional factors may be administered before, simultaneously with, or after the administration of the insulin-producing cells or precursors of insulin-producing cells. For example, the cells (either precursor cells or fully differentiated insulin-producing cells) and one or more growth factors may be included in the same device or encapsulation for implantation.

The cells administered to a diabetic patient, either a type 1 or type 2 diabetic patient, may be generated from autologous sources, e.g., a co-culture of MSCs and pancreatic cells obtained from the patient being treated, in order to avoid the immune rejection that would accompany an allogeneic transplant. In the case of treating a type I diabetic with autologous cells, the prevention of autoimmune destruction of the cells may require some immune intervention. However, where cells of autologous sources are not available, insulin-producing cells or precursors thereof produced from allogenic or xenogenic cells may also be used. In this instance, it may be useful to encapsulate the insulin-producing cells or precursors of insulin-producing cells in a capsule that is permeable to the endocrine hormones, including insulin, glucagon, somatostatin and other pancreas produced factors, yet impermeable to immune humoral factors and cells. Preferably the encapsulant is hypoallergenic, is easily and stably situated in a target tissue, and provides added protection to the implanted structure.

The cells administered to a patient determined to be predisposed to develop diabetes may be generated from autologous sources, e.g., a co-culture of MSCs and pancreatic cells obtained from the patient being treated, in order to avoid the immune rejection that would accompany an allogeneic transplant. In the case of treating a type I diabetic with autologous cells, the prevention of autoimmune destruction of the cells may require some immune intervention. However, where cells of autologous sources are not available, insulin-producing cells or precursors thereof produced from allogenic or xenogenic cells may also be used. In this instance, it may be useful to encapsulate the insulin-producing cells or precursors of insulin-producing cells in a capsule that is permeable to the endocrine hormones, including insulin, glucagon, somatostatin and other pancreas produced factors, yet impermeable to immune humoral factors and cells. Preferably the encapsulant is hypoallergenic, is easily and stably situated in a target tissue, and provides added protection to the implanted structure.

The amount of cells that should be used in implantation depends on a number of factors including the patient's condition and response to the therapy, and may be determined by a physician.

In yet a further aspect of the present invention, a method of treating a diabetic subject is provided wherein mesenchymal stem cells are administered to the subject to promote the generation of insulin-producing cells in the subject.

According to the present invention, the administered mesenchymal stem cells can supply the signals that promote the proliferation and differentiation of precursors of insulin-producing cells that are present in the subject. Alternatively or additionally, the administered mesenchymal stem cells, upon interaction with cells in the pancreas of the subject, can develop or transdifferentiate into insulin-producing cells.

Mesenchymal stem cells used for administration may be derived from the subject being treated (i.e., autologous). Allogenic and xenogenic mesenchymal stem cells may also be used. The cells may be administered to the subject by local injection into the pancreas or into a site proximate to the pancreas. Alternatively, the cells may be implanted in the pancreas or at a site proximate to the pancreas of the subject.

In yet a further aspect of the present invention, a method of treating a subject determined to be predisposed to develop diabetes is provided wherein mesenchymal stem cells are administered to the subject to promote the generation of insulin-producing cells in the subject.

According to the present invention, the administered mesenchymal stem cells can supply the signals that promote the proliferation and differentiation of precursors of insulin-producing cells that are present in the subject. Alternatively or additionally, the administered mesenchymal stem cells, upon interaction with cells in the pancreas of the subject, can develop or transdifferentiate into insulin-producing cells.

Mesenchymal stem cells used for administration may be derived from the subject being treated (i.e., autologous). Allogenic and xenogenic mesenchymal stem cells may also be used. The cells may be administered to the subject by local injection into the pancreas or into a site proximate to the pancreas. Alternatively, the cells may be implanted in the pancreas or at a site proximate to the pancreas of the subject.

The present invention is further illustrated but not limited by the following examples.

### EXAMPLE 1

Design: PANC-1 cells (American Type Culture Collection, Manassas, VA) were started at passage 3. Cells (1−5 ×10ˆ5) were cultured in Dulbeccos Minimal Essential Media (DMEM) containing 10% fetal bovine serum (FBS) in 6-well and 10 cm tissue culture treated dished until 70% confluency was achieved. In an attempt to induce cell differentiation, the serum containing media (SCM) was removed and the cells were exposed for 60-120 seconds to 0.05% trypsin (Cellgro, Mediatech, Hemdon, VA) at 25° C., to loosen but not to detach the cells from their extracellular matrix (ECM), and then cultured in serum-free media with the DMEM/F12 medium containing 17.5 mM glucose, 1-2% bovine serum albumin (BSA), insulin, transferrin, and selenium (ITS-GIBCO, Long Island, N.Y.).

Results: Islet-like cell clusters were formed after the induction with the ITS media after 2 days.

### EXAMPLE 2

Design: Human MSCs from Clontech (Palo Alto, CA), passage 4-6, and Pancl cells from ATCC, passage 2-6, were used in the experiment. 5 ×10ˆ5 cells for both cells were seeded in 10 cm tissue culture treated dishes in a 1:1 combination media of DMEM containing 10% FBS and a MSC growth media purchased from CAMBREX (Walkersville, MD). After 2-3 days of culture, the media was replaced by the induction media containing1% ITS, 2% BSA in DMEM. The proteasome inhibitor, Lactocystin, was added into the media at the final concentration of 100 μM to potentiate the differentiation for 10 days.

Results: No formation of clusters or expression of insulin was observed.

### EXAMPLE 3

Design: Human MSCs from Clontech, passage 4-6, and Pancl cells from ATCC, passage 2-6, were used in the experiment. 5 ×10ˆ5 cells for both cells were seeded in 10 cm tissue culture treated dishes in a 1:1 media of DMEM containing 10% FBS, and a MSC growth media purchased from CAMBREX. After 2-3 days of culture, the media was replaced by the induction media containing 1% ITS, 2% BSA in DMEM for 10 days. A growth factor cocktail containing bFGF, EGF, Exendin-4 and Nicotinamide, was added into the media to potentiate the differentiation.

Results: There was an increase of islet-like cell cluster formation in the co-culture system with the cocktail of growth factors, as compared to a co-culture system without the cocktail of growth factors.

### EXAMPLE 4

Design: Human MSCs from Clontech, passage 4-6, and Pancl cells from ATCC, passage 2-6, were used in the experiment. 5 ×10ˆ5 cells for both cells were seeded in 10 cm tissue culture treated dishes in a 1:1 media of DMEM with 10% FBS, and MSC growth media purchased from CAMBREX. After 2-3 days of culture, the media was replaced by the induction media containing 1% ITS, 2% BSA in DMEM for 10 days. Growth factors including bFGF combined with, EGF, GLP-1 (Exendin-4), Nicotimamide, and p38 kinase inhibitor were added into the media to potentiate the differentiation.

Results: The results show that more islet-like cell clusters were formed in the co-culture system, suggesting that bFGF, combined with EGF, GLP-1 (Exendin-4), Nicotimamide, and p38 kinase inhibitor may facilitate the cluster formation.

### EXAMPLE 5

Isolation ofMSCsfrom Rats: Rats were sacrificed by cervical dislocation under anesthesia with isoflurane and were dissected to obtain the femurs and tibias. The bones were cut at the ends to gain access to the marrow cavity. The marrow was flushed out of each bone using phosphate buffered saline (PBS) in a syringe with a 23 to 25-gauge needle. The marrow and PBS were collected in Petri dish. A single-cell suspension was prepared by repeatedly drawing and dispensing the marrow suspension through the needle and the syringe. The cells were collected into a 50 mL tube containing DMEM and 10% FBS medium. The tube was centrifuged at 800 rpm for 2 minutes at 4° C. Following centrifugation the supernatant was removed and the pellet resuspended in DMEM medium. After being washed three times, the cells were cultured in a 10 cm culture dish containing DMEM with 2-5% FBS for two days, followed by a change of medium to eliminate most of the non-adherent hematopoietic cells. The cells were passaged 7 days after initial seeding at 75% confluency using trypsin/EDTA. After one to two passages, the MSCs were ready for use in a co-culture with pancreatic cells.

### EXAMPLE 6

Isolation ofprogenitorsfrom ratpancreas: Rats were anaesthetized with Nembutal via IP injection. Following confirmation of anesthesia with toe pinch, cervical dislocation was performed. The site of incision was cleaned with 70% alcohol solution. A full length V-incision was made with scissors from groin past the breastbone. After moving the liver to the side, the common bile duct and pancreas were exposed. Using a hemostat or forceps, the bile duct was clamped at where it met the duodenum. With a 21G1/2 needle syringe, 10 cc HBSS medium with 0.25 mg/ml Liberase® was slowly injected into the bile duct. The pancreas was carefully cut away from all connective tissue, and then placed in a 50-ml tube on ice. The tube was then placed in a water bath at 37° C. for a period of about 18 to 25 minutes. Afterwards, the tube was removed from the water bath and shaken vigorously for 5 seconds. To stop the digestion, the sample was washed by centrifugation at 1000 rpm for 2 min at 4° C., followed by aspirating the supernatant down to the 10 ml mark and adding cold quenching buffer (HBSS with 10% FBS) to a final volume of 50 ml. The wash was repeated and the supernatant was aspirated completely, and the cell pellet was resuspended in the cold quenching buffer of 15 ml. The digested tissue was then poured through a stainless steel mesh with a finnel into a 150 ml bottle. The mesh was rinsed with a modified HBSS medium giving a final volume of 100 ml. The tissue suspension was divided into two 50-ml tubes, which were subjected to centrifugation at 1000 rpm for 2 min at 4° C. After aspirating the supernatant, the 1.108-FICOLL Polysucrose solution layer of 10 ml was added to each of the tubes. The tubes were capped and then vortexed. Afterwards, 5 ml each of the next three layers were slowly added to the tubes to form the gradient: 1.096, 1.069 and 1.037-FICOLL Polysucrose solutions (of different densities). The tubes were subjected to centrifugation at 2000 rpm for 10 minutes at 4° C. The centrifuge rotor did not have the brake engages for the final centrifugation step. Five fractions were obtained from the gradient: cells above the first Ficoll interfaces, cells across the first Ficoll interface, cells across the second interface, cells across the third interface, and cells in the pellet. Cell fractions were collected separately and transferred to 50-ml tubes containing 35 to 40 ml cold quenching buffer. The tubes were then filled up with the cold quenching buffer and centrifuged at 1200 rpm for 3 minutes at 4° C. The supernatant was aspirated down to the 10 ml mark and the cold quenching buffer was added to the 50 ml mark. After repeating the wash two more times (the first time at 1200 rpm for 2 min. and the second time at 1200 rpm for 1 min), the supernatant was aspirated and the pellet was resuspended in the CMRL-1066 medium of 10 ml at room temperature, supplemented with P/S, 10% of FBS, 2 mM of L-glutamine. The cell suspensions were then transferred to 10-cm dishes for culture at 37° C. and 5% CO₂.
Typically the cells contained within each fraction were as follows:
above the 1.037 gradient—cell debris, fat, membrane balls, degranulated acinar;
at the 1.037/1.069 interface—cell debris, membrane balls, degranulated acinar;
at the 1.069/1.096 interface—greater than 50% islets, cell debris, less granulated acinar, membrane balls, duct;
at the 1.096/1.108 interface—less than 50% islets, acinar, duct;
below the 1.108 gradient—90% acinar, duct, less than 1% islet.
   Publications cited throughout this document are hereby incorporated by reference in their entirety. Although the various aspects of the invention have been illustrated above by reference to examples and preferred embodiments, it will be appreciated that the scope of the invention is defined not by the foregoing description, but by the following claims properly construed under principles of patent law.

What is claimed is:

1. A method of promoting the growth of pancreatic cells, comprising obtaining a population of pancreatic cells, culturing said population of pancreatic cells in the presence of mesenchymal stem cells to allow the growth and proliferation of the pancreatic cells in said cultured cell population.

2. The method according to claim 1, wherein said population of pancreatic cells is prepared from a mammalian pancreas or from a pancreatic cell line.

3. The method of claim 1, wherein said mesenchymal stem cells are prepared from the bone marrow, umbilical cord blood, amniotic sac and fluid, placenta, skin, fat, muscle, vasculature, liver, pancreas, or peripheral blood of a mammal.

4. The method of any one of claims 1-3, wherein the pancreatic cells are undifferentiated pancreatic cells.

5. The method of claim 4, wherein said undifferentiated pancreatic cells are characterized by the lack of expression of at least one marker specific to differentiated pancreatic cells.

6. A method of promoting the generation and/or proliferation of insulin-producing cells, comprising culturing a population of pancreatic cells in the presence of mesenchymal stem cells to allow the generation and/or proliferation of insulin-producing cells.

7. The method according to claim 6, wherein said population of pancreatic cells is prepared from a mammalian pancreas or from a pancreatic cell line.

8. The method of claim 6, wherein said population of pancreatic cells comprises undifferentiated cells.

9. The method of claim 6, wherein said mesenchymal stem cells are prepared from the bone marrow, umbilical cord blood, amniotic sac and fluid, placenta, skin, fat, muscle, vasculature, liver, pancreas, or peripheral blood of a mammal.

10. A method of generating insulin-producing cells, comprising culturing a pancreatic cell population in the presence of mesenchymal stem cells to allow the generation and/or proliferation of precursors of insulin-producing cells, and developing the precursors into insulin-producing cells.

11. The method of claim 10, wherein the precursors are differentiated into insulin-producing cells in a cell culture.

12. The method of claim 11, wherein mesenchymal stem cells are provided in the cell culture to promote the differentiation of the precursors into insulin-producing cells.

13. The method of claim 10, wherein said precursors are further differentiated into insulin-producing cells in a mammal.

14. The method of claim 13, wherein mesenchymal stem cells are also supplied to said mammal to promote the differentiation of the precursors into insulin-producing cells.

15. The insulin-producing cells made by any method of claims 6-14.

16. A method of promoting the generation and/or proliferation of precursors of insulin-producing cells, comprising culturing a population of pancreatic cells in the presence of mesenchymal stem cells to allow the generation and/or proliferation of precursors of insulin-producing cells.

17. The method of claim 16, where said precursors are characterized by the lack of expression of at least one marker specific to differentiated pancreatic cells and by the expression of at least one marker specific to the beta cell lineage.

18. The method of claim 17, where said marker specific to differentiated pancreatic cells is selected from the group consisting of insulin, glucagon, somatostatin, pancreatic polypeptide, amylase, lipase, cytokeratin, PDX-1 (pancreatic and duodenal homeobox gene-1), NGN-3 (neurogenin-3), Hlxb9, Nkx6.1, Nkx2.2, Mafk, Isl1, NeuroD, HNF1α & β, HNF4α, HNF6, Pax4, and Pax6.

19. The method of claim 16, wherein said marker specific to the beta cell lineage is selected from the group consisting of insulin, glut 2, PDX-1 (pancreatic and duodenal homeobox gene-1), NGN-3 (neurogenin-3), Hlxb9, Nkx6.1, Nkx2.2, MafA, Isl1, NeuroD, HNF1α & β, HNF4 α, HNF6, Pax4, and Pax6.

20. The precursors of insulin-producing cells made by any method of claims 16-19.

21. A method of treating a diabetic subject or a subject determined to be predisposed to develop diabetes, comprising administering to the subject insulin-producing cells or precursors thereof made according to claim 6 or 16.

22. The method of claim 21, wherein the insulin-producing cells or precursors thereof are of an autologous, allogenic or xenogenic origin.

23. The method of claim 21, wherein the insulin-producing cells or precursors thereof are administered by local injection or implantation in the pancreas or at a site proximate to the pancreas of the subject.

24. The method of claim 21, further comprising administering mesenchymal stem cells to the subject.

25. A method of treating a diabetic subject, comprising administering mesenchymal stem cells to the subject, thereby promoting the generation and/or proliferation of insulin-producing cells in the subject.

26. A method of treating a subject determined to be predisposed to develop diabetes, comprising administering mesenchymal stem cells to the subject, thereby promoting the generation and/or proliferation of insulin-producing cells in the subject

27. A method of promoting the generation and/or proliferation of insulin-producing cells, comprising culturing a population of pancreatic cells in the presence of mesenchymal stem cells to allow the transdifferentiation of mesenchymal stem cells into insulin-producing cells.

28. The method of any one of claim 1, 6, 16 or 25 further comprising growth factors.
